(19) United States
(12) Patent Application Publication
(10) Pub. No.: US 2013/0085359 A1
(43) Pub. Date: Apr. 4, 2013

(54) FLEXIBLE MICROELECTRODE FOR DETECTING NEURAL SIGNALS AND A METHOD OF FABRICATING THE SAME

(71) Applicants: Da-Jeng Yao, Hsinchu City (TW); Chang-Hsiao Chen, New Taipei City (TW)

(72) Inventors: Da-Jeng Yao, Hsinchu City (TW); Chang-Hsiao Chen, New Taipei City (TW)

(21) Appl. No.: 13/359,913

(22) Filed: Jan. 27, 2012

(30) Foreign Application Priority Data
Sep. 30, 2011 (TW) 100135438

(51) Int. Cl. A61B 5/04; B32B 37/14; B32B 37/02; B82Y 40/00

(52) U.S. Cl. 600/372; 156/185; 977/842

(57) ABSTRACT

A flexible microelectrode for detecting neural signals and a method of fabricating the same are disclosed. The method comprises steps: growing a graphene electrode layer on a temporary substrate; growing a flexible substrate on the graphene electrode layer and patterning the flexible substrate; removing the temporary substrate but preserving the graphene electrode layer and the flexible substrate to form an electrode body; and using an insulating layer to wrap the electrode body but exposing a bio-electrode end to contact a living body and detect the signals thereof. The graphene electrode layer features high electric conductivity, high biocompatibility and low noise. The flexible substrate is bendable. Thus is improved the adherence of the skin tissue to the bio-electrode end and decreased the likelihood of skin inflammation.

### FIELD OF THE INVENTION

The present invention relates to a flexible electrode, particularly to a flexible microelectrode for detecting neural signals and a method of fabricating the same.

### BACKGROUND OF THE INVENTION

The brain and the nervous system are neural networks formed of numerous cross-linked neurons. It is very important to understand the operation of the nervous system for diagnosis, therapy and prosthesis design of neural diseases. Probes can easily pierce the skin and detect electrophysiological signals in vivo, so they can also function as a medium between analog physiological signals and digital signals.

The abovementioned probe is an electrode of a biomicroelectromechanical system, which should be able to conduct very weak nerve current. Therefore, the electrode must have high electric conductivity. Further, the electrode should have high biocompatibility so that cells can adhere thereto and survive thereon. The heartbeat and breathing of an animal or a human being will cause the cells or tissue on the body surface thereof to pulsate. When a probe is directly applied to the body surface, the pulsation will cause tiny friction between the probe and the cells. The tiny friction may accelerate skin inflammation. Therefore, flexibility is necessary for an electrophysiological electrode.

A prior art disclosed an electrode having a carbon nanotube interface, wherein the surface of the carbon nanotubes has abundant carboxyl groups to effectively reduce impedance between the electrode and the tissue fluid, whereby is achieved better measurement quality. A U.S. patent of publication No. 20100268055, a “Self-Anchoring MEMS Intrafascicular Neural Electrode”, disclosed a method for using the same to detect, record, and stimulate the activity of the nervous system and the peripheral nerve tracts. However, the conductivity of the electrode disclosed in the prior art still generates much noise. Therefore, the prior art cannot provide required sensitivity for neural signal detection. Further, the biocompatibility and flexibility of the prior art should be improved.

### SUMMARY OF THE INVENTION

The primary objective of the present invention is to provide an electrode structure having high biocompatibility, flexibility and electric conductivity.

To achieve the abovementioned objective, the present invention proposes a method of fabricating a flexible microelectrode for detecting neural signals, which comprises steps:

S1: growing a graphene electrode layer on a temporary substrate;

S2: growing a flexible substrate on one surface of the graphene electrode layer, which is far away from the temporary substrate;

S3: removing the temporary substrate and preserving the graphene electrode layer and the flexible substrate to form an electrode body, wherein the electrode body has a bio-electrode end and an interface-connection end; and

S4: using an insulating layer to wrap the electrode body but expose the bio-electrode end.

The flexible microelectrode fabricated according to the abovementioned method comprises an electrode body and an insulating layer. The electrode body includes a flexible substrate and a graphene electrode layer. The electrode body has a bio-electrode end and an interface-connection end. The insulating layer warps the graphene electrode layer but reveal the bio-electrode end.

The graphene electrode layer is a 2D graphite structure having very high electric conductivity. Further, graphene has biocompatibility much superior to that of ordinary metallic electrodes. Besides, the flexible substrate enables the electrode to bend, and the insulating layer protects the electrode from external interference.

### BRIEF DESCRIPTION OF THE DRAWINGS

FIGS. 1A-1F are sectional views schematically showing steps of fabricating a flexible microelectrode for detecting neural signals according to one embodiment of the present invention;

FIGS. 2A-2C are perspective views schematically showing steps of fabricating a flexible microelectrode for detecting neural signals according to one embodiment of the present invention;

FIG. 3A is a photograph showing the state that neural cells adhere to glass;

FIG. 3B is a photograph showing the state that neural cells adhere to graphene;

FIG. 3C is a photograph showing the state that neural cells adhere to graphene fabricated by a steam plasma method according to one embodiment of the present invention; and

FIG. 4 shows the signals and SNR obtained via a flexible microelectrode fabricated according to one embodiment of the present invention.

### DETAILED DESCRIPTION OF THE PREFERRED EMBODIMENTS

The technical contents of the present invention are described in detail in cooperation with the drawings below.

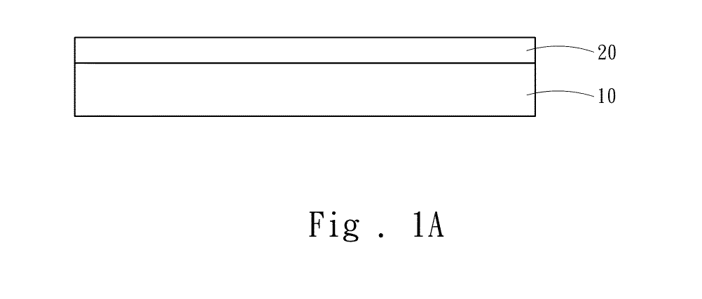

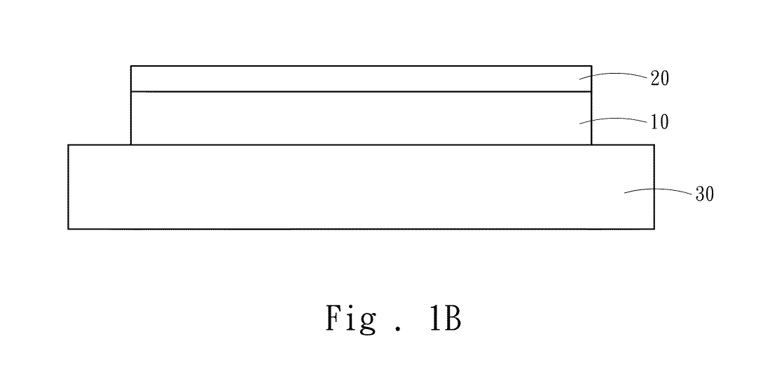

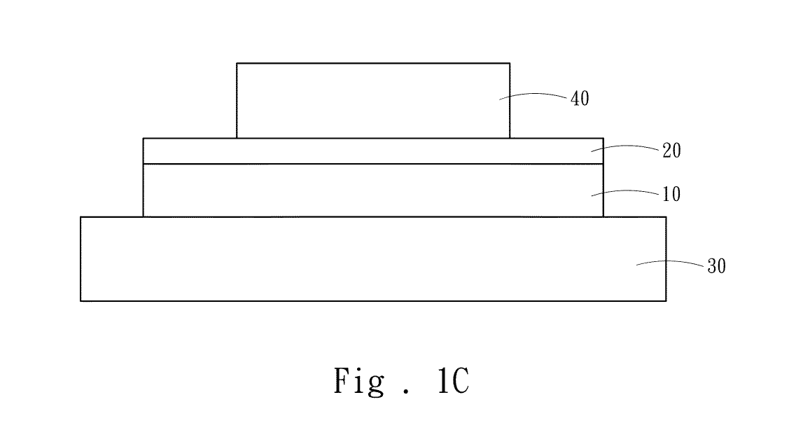

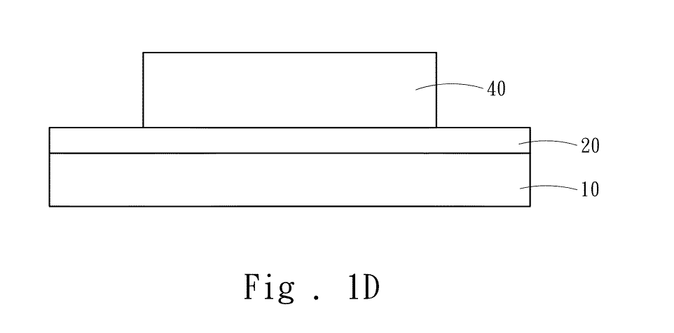

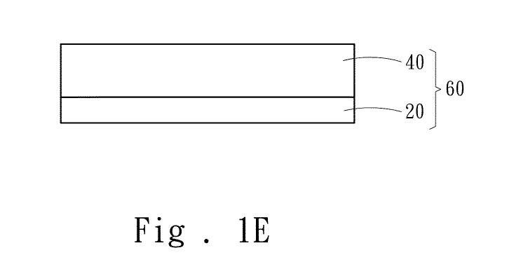

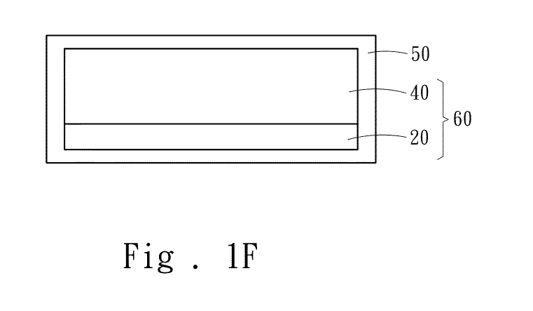

Refer to FIGS. 1A-1F sectional views schematically showing steps of fabricating a flexible microelectrode for detecting neural signals according to one embodiment of the present invention.

In Step S1, a graphene electrode layer 20 is grown on a temporary substrate 10, as shown in FIG. 1A. The graphene electrode layer 20 is grown on the temporary substrate 10 with a CVD (Chemical Vapor Deposition) method. In details, the temporary substrate 10 is made of copper; the temporary substrate 10 is annealed in a tube furnace filled with a gas mixture of hydrogen and argon to remove the organic substances and oxides thereon; then the tube furnace is filled with methane and maintained at a temperature of 1000° C. to form the graphene electrode layer 20 on the temporary substrate 10.

In order to provide the succeeding steps with a more stable environment, the present invention further comprises Step Al.

In Step A1, a transfer-printing substrate 30 is grown on one surface of the temporary substrate 10, which is far away from the graphene electrode layer 20, as shown in FIG. 1B. In one embodiment, the transfer-printing substrate 30 is made of PDMS (Polydimethylsiloxane). In one embodiment, the transfer-printing substrate 30 is grown on the surface of the temporary substrate 10 with a spin-coating method.

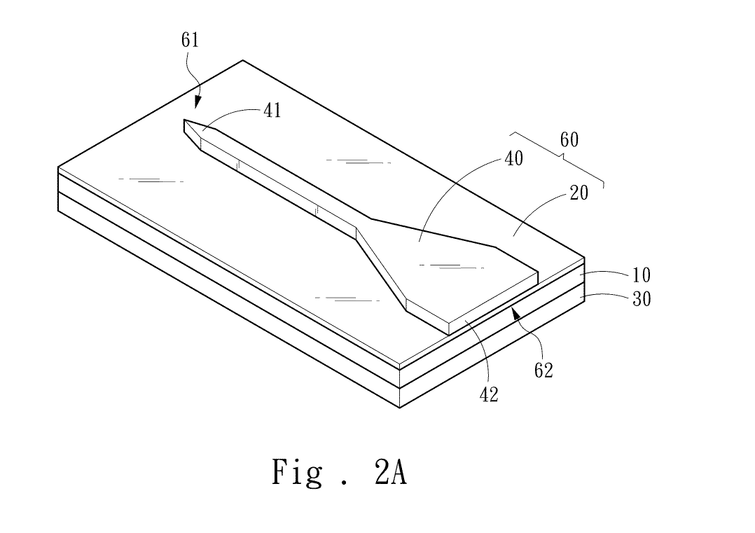

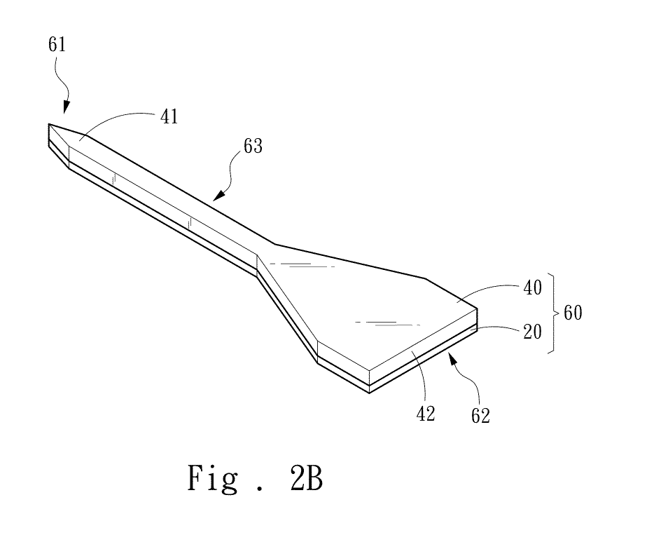

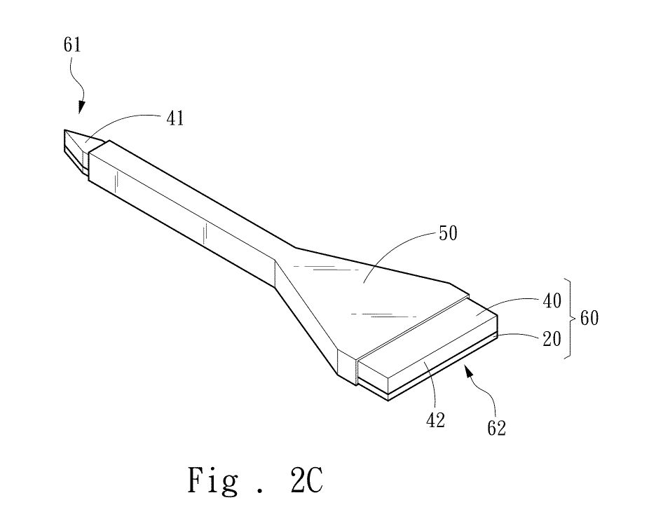

In Step S2, a flexible substrate 40 is grown on one surface of the graphene electrode layer 20, which is far away from the temporary substrate 10, as shown in FIG. 1C and FIG. 2A. In one embodiment, the flexible substrate 40 is formed with a spin-coating method. In one embodiment, the flexible substrate 40 is made of an epoxy-based negative photoresist, such as SU-8. SU-8 can be fabricated into a thick flexible layer. Therefore, SU-8 can be fabricated into a flexible substrate 40 having high insulativity and flexibility. Next, use a patterning process to form a first end 41 and a second end 42 opposite to the first end 41 on the flexible substrate 40. The first end 41 gradually contracts toward a direction far away from the second end 42. The second end 42 gradually expands toward a direction far away from the first end 41. In one embodiment, the first end 41 is fabricated to have a needle-like shape, and the second end 42 is fabricated to have a plate-like shape. However, the first end 41 and second end 42 of the flexible substrate 40 may be fabricated to have other shapes according to practical requirement.

In Step A2, the transfer-printing substrate 30 is removed after the flexible substrate 40 is completed, as shown in FIG. 1D.

In Step S3, the temporary substrate 10 is removed with oxide of iron ion, as shown in FIG. 1E. Next, the flexible substrate 40 is applied as a mask to perform a patterning process on the graphene electrode layer 20 to fabricate the graphene electrode layer 20 to have a shape corresponding to the shape of the flexible substrate 20, as shown in FIG. 2B. Thus is formed an electrode body 60 containing the graphene electrode layer 20 and the flexible substrate 40. The electrode body 60 has a bio-electrode end 61, an interface-connection end 62 and a middle region 63 between the bio-electrode end 61 and the interface-connection end 62. The bio-electrode end 61 gradually contracts toward a direction far away from the interface-connection end 62. The interface-connection end 62 gradually expands toward a direction far away from the bio-electrode end 61. Similar to the first end 41, the bio-electrode end 61 is fabricated to have a needle-like shape. Similar to the second end 42, the interface-connection end 62 is fabricated to have a plate-like shape. In the abovementioned steps, the flexible substrate 40 and the graphene electrode layer 20 are patterned in sequence. However, the abovementioned steps are only to exemplify the present invention. The present invention is not limited by the abovementioned steps. In a practical process, the flexible substrate 40 and the graphene electrode layer 20 may be patterned at the same time. The bio-electrode end 61 will contact an animal or a human being (not shown in the drawings) to detect signals. The interface-connection end 62 transmits the signals to a test device (not shown in the drawings).

In Step S4, an insulating layer 50 is applied to wrap the middle region 63 of the electrode body but expose the bio-electrode end 61. The exposed bio-electrode end 61 will contact an animal or a human being and detect the signals thereof. In one embodiment, the insulating layer 50 is made of PDMS. The interface-connection end 62 may be exposed from or wrapped by the insulating layer 50 according to the test device to be connected with the interface-connection end 62.

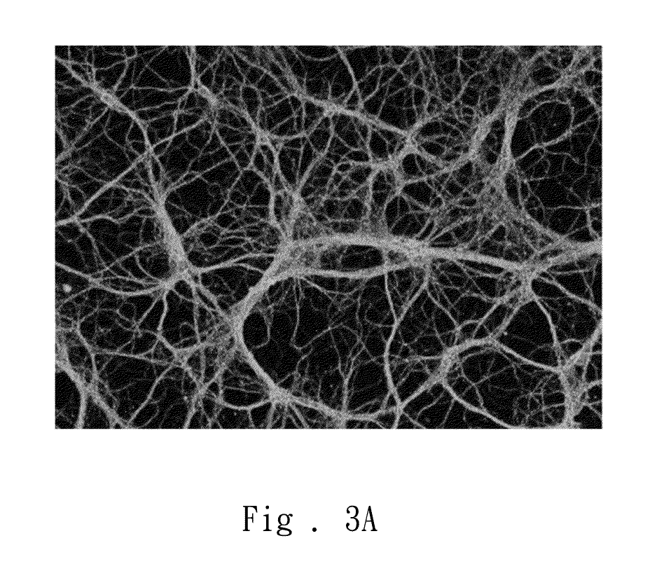

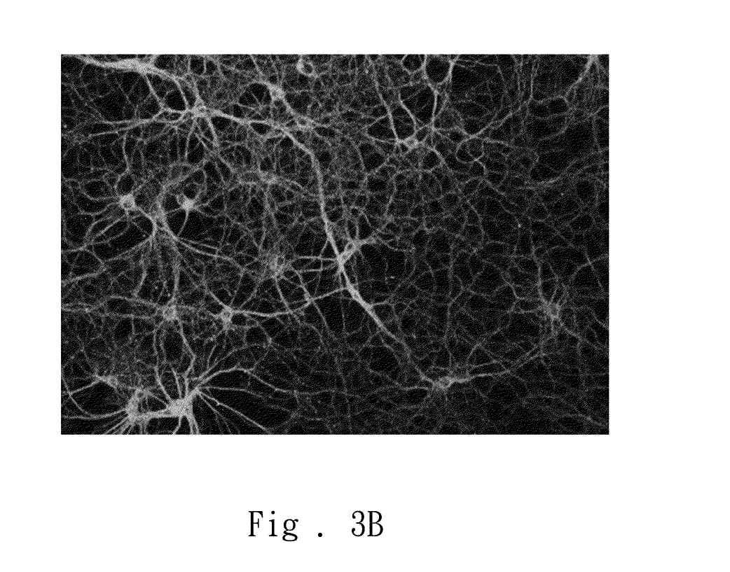

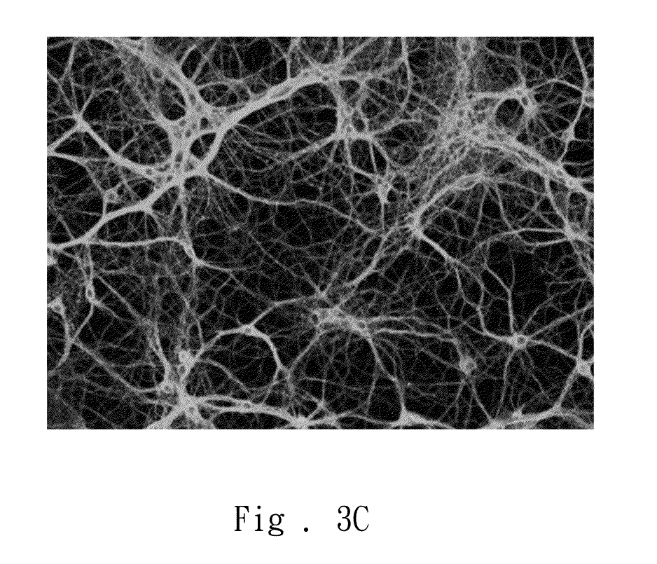

Refer to FIG. 3A a photograph showing the state that neural cells adhere to glass. Generally speaking, cells can develop on and adhere to glass most optimally. The density of neural cells on glass reaches as high as 74.6 per square millimeter. However, the electric conductivity of glass is poor. Contrarily, the graphene has an electric conductivity of over 15,000 cm²v⁻¹s⁻¹. From FIG. 3B, it is learned that the density of neural cells on graphene is about 61 per square millimeter. Nevertheless, the density of neural cells on an ordinary metal is only about 20 per square millimeter. Therefore, the graphene electrode layer 20 of the present invention outperforms ordinary metals in biocompatibility. In addition to general CVD methods, the graphene electrode layer 20 may be formed on the temporary substrate 10 with a steam plasma method to increase the electrochemical adhesion and biocompatibility of the graphene electrode layer 20, whereby the density of neural cells on the graphene electrode layer 20 can reach as high as 77.4 per square millimeter, as shown in FIG. 3. In such a case, the level of biocompatibility of the graphene electrode layer 40 is identical to that of glass.

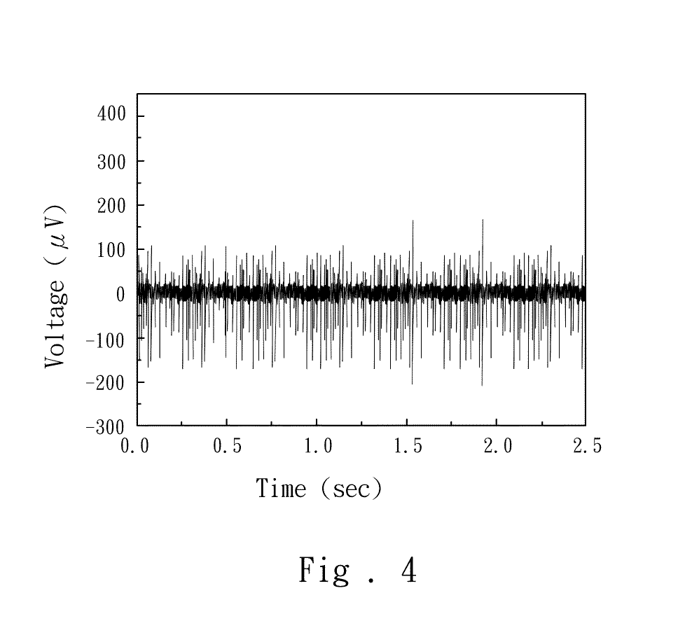

Refer to FIG. 4. The signal obtained with the flexible microelectrode of the present invention has SNR (Signal to Noise Ratio) as high as 35.38 dB. Therefore, the present invention can achieve higher signal sensitivity and obtain better measurement results.

The present invention also discloses a flexible microprobe for detecting neural signals, which comprises an electrode body 60 and an insulating layer 50. The electrode body 60 includes a flexible substrate 40 and a graphene electrode layer 20 formed on the flexible substrate 40. The electrode body 60 has a bio-electrode end 61 and an interface-connection end 62. The flexible substrate 40 is made of a polymeric material SU-8. The shape of the graphene electrode layer 20 is corresponding to that of the flexible substrate 40. The insulating layer 50 wraps the graphene electrode layer 20 but reveal the bio-electrode end 61. In one embodiment, the interface-connection end 62 is also exposed from the insulating layer 50 for connecting with a test device. In one embodiment, the insulating layer 50 is made of PDMS.

In conclusion, the graphene electrode layer 20 is a 2D graphite structure so it has very high electric conductivity. Further, graphene has biocompatibility much superior to that of ordinary metallic electrodes. Besides, the flexible substrate 40 enables the electrode to bend, and the insulating layer 50 protects the electrode from external interference lest tiny vibration cause friction and accelerate inflammation. Furthermore, the present invention also discloses a method of using a steam plasma method of fabricating the graphene electrode layer 20, whereby to promote the biocompatibility of the graphene electrode layer 20. Therefore, the microelectrode of the present invention features flexibility, high biocompatibility and high electric conductivity simultaneously.

The present invention possesses utility, novelty and non-obviousness and meets the condition for a patent. Thus, the Inventors file the application for a patent. It is appreciated if the patent is approved fast.

The embodiments described above are only to exemplify the present invention but not to limit the scope of the present invention. Any equivalent modification or variation according to the scope of the present invention is to be also included within the scope of the present invention.

What is claimed is:

1. A method of fabricating a flexible microelectrode for detecting neural signals, comprising
Step S1: growing a graphene electrode layer on a temporary substrate;
Step S2: growing a flexible substrate on one surface of the graphene electrode layer, which is far away from the temporary substrate;
Step S3: removing the temporary substrate but preserving the graphene electrode layer and the flexible substrate to form an electrode body having a bio-electrode end, an interface-connection end and a middle region between the bio-electrode end and the interface-connection end; and
Step S4: using an insulating layer to wrap the middle region but expose the bio-electrode end.

2. The method of fabricating a flexible microelectrode for detecting neural signals according to claim 1, wherein the graphene electrode layer is grown on the temporary substrate with a CVD (Chemical Vapor Deposition) method.

3. The method of fabricating a flexible microelectrode for detecting neural signals according to claim 1, wherein the flexible substrate is made of a polymeric material SU-8, and wherein the temporary substrate is made of copper.

4. The method of fabricating a flexible microelectrode for detecting neural signals according to claim 1, wherein in Step S1, the graphene electrode layer is formed on the temporary substrate with a steam plasma method.

5. The method of fabricating a flexible microelectrode for detecting neural signals according to claim 1, wherein in Step S2, the flexible substrate is formed on the graphene electrode layer with a spin-coating method.

6. The method of fabricating a flexible microelectrode for detecting neural signals according to claim 1, wherein in Step S4, the insulating layer wraps the middle region but expose the interface-connection end of the electrode body.

7. The method of fabricating a flexible microelectrode for detecting neural signals according to claim 1, wherein in Step S4, the insulating layer is made of PDMS (Polydimethylsiloxane).

8. The method of fabricating a flexible microelectrode for detecting neural signals according to claim 1, wherein in Step S3, a patterning process is used to make the bio-electrode end of the electrode body gradually contract toward a direction far away from the interface-connection end and make the interface-connection end of the electrode body gradually expand toward a direction far away from the bio-electrode end.

9. A flexible microelectrode for detecting neural signals, comprising:
an electrode body including a flexible substrate and a graphene electrode layer formed on the flexible substrate and having a bio-electrode end and an interface-connection end; and
an insulating layer wrapping the graphene electrode layer but exposing the bio-electrode end.

10. The flexible microelectrode for detecting neural signals according to claim 9, wherein the electrode body also has a middle region arranged between the bio-electrode end and the interface-connection end and wrapped by the insulating layer.

11. The flexible microelectrode for detecting neural signals according to claim 9, wherein the flexible substrate is made of a polymeric material SU-8.

12. The flexible microelectrode for detecting neural signals according to claim 9, wherein the insulating layer is made of PDMS (Polydimethylsiloxane).

13. The flexible microelectrode for detecting neural signals according to claim 9, wherein the interface-connection end is exposed from the insulating layer.

14. The flexible microelectrode for detecting neural signals according to claim 9, wherein the bio-electrode end of the electrode body gradually contracts toward a direction far away from the interface-connection end.

15. The flexible microelectrode for detecting neural signals according to claim 9, wherein the interface-connection end of the electrode body gradually expands toward a direction far away from the bio-electrode end.
